(19) United States
(12) Patent Application Publication
(10) Pub. No.: US 2026/0091096 A1
(43) Pub. Date: Apr. 2, 2026

(54) MEASURING THE PROGRESS OF CARDIAC XENOGRAFT IMMUNE REJECTION

(71) Applicant: Mayo Foundation for Medical Education and Research, Rochester, MN (US)

(72) Inventors: Guerard W. Byrne, Allentown, NJ (US); Christopher G.A. McGregor, Rochester, MN (US)

(21) Appl. No.: 19/329,042

(22) Filed: Sep. 15, 2025

Related U.S. Application Data
(62) Division of application No. 17/459,410, filed on Aug. 27, 2021, now abandoned.
Application No. 17/459,410 is a division of application No. 15/494,976, filed on Apr. 24, 2017, now Pat. No. 11,141,470.
Application No. 15/494,976 is a division of application No. 13/696,478, now Pat. No. 9,642,899.
(60) Provisional application No. 61/332,127, filed on May 6, 2010.

(51) Int. Cl. A61K 39/00; A01K 67/0276; A61K 35/34; A61K 38/00; C12N 15/85

(52) U.S. Cl. CPC A61K 39/001; A01K 67/0276; A61K 35/34; C12N 15/8509; A01K 2207/30; A01K 2227/108; A01K 2267/02; A01K 2267/025; A61K 38/00

(57) ABSTRACT

This document provides methods and materials involved in reducing cardiac xenograft rejection. For example, methods and materials for preparing transgenic pigs expressing reduced or no endogenous Sd^{a} or SDa-like glycans derived from the porcine β1,4 N-acetyl-galactosaminyl transferase 2 (B4GALNT2) glycosyltransferase and/or reduced or no endogenous α-Gal antigens, methods and materials for modifying the xenograft recipient's immunological response to non-Gal antigens (e.g. CD46, CD59, CD9, PROCR, and ANXA2) to reduce cardiac xenograft rejection, and methods and materials for monitoring the progress of xenotransplant immunologic rejection are provided.

### CROSS-REFERENCE TO RELATED APPLICATIONS

This application is a divisional of U.S. application Ser. No. 17/459,410, filed on Aug. 27, 2021, which is a divisional of U.S. application Ser. No. 15/494,976, filed on Apr. 24, 2017 (now U.S. Pat. No. 11,141,470), which is a divisional of U.S. application Ser. No. 13/696,478, filed Jan. 17, 2013 (now U.S. Pat. No. 9,642,899), which is a National Stage application under 35 U.S.C. § 371 of International Application No. PCT/US2011/031976, filed Apr. 11, 2011, which claims the benefit of U.S. Provisional Application Ser. No. 61/332,127, filed May 6, 2010. The disclosure of the prior applications is considered part of (and are incorporated by reference in) the disclosure of this application.

### STATEMENT AS TO FEDERALLY SPONSORED RESEARCH

This invention was made with government support under AI066310 awarded by the National Institutes of Health. The government has certain rights in the invention.

### STATEMENT REGARDING SEQUENCE LISTING

This application contains a sequence listing that has been submitted electronically as an XML file named “07039-0993004_SL_ST26.xml.” The XML file, created on Sep. 12, 2025, is 46,340 bytes in size. The material in the XML file is hereby incorporated by reference in its entirety.

### BACKGROUND

### 1. Technical Field

This document relates to methods and materials involved in reducing cardiac xenograft rejection. For example, this document provides methods and materials for preparing transgenic pigs expressing reduced or no endogenous Sd^{a}, reduced or no endogenous SDa-like glycans derived from the porcine β1,4 N-acetyl-galactosaminyl transferase 2 (B4GALNT2) glycosyltransferase, and/or reduced or no endogenous α-Gal antigens, methods and materials for modifying a xenograft recipient's immunological response to non-Gal antigens (e.g. CD46, CD59, CD9, porcine endothelial cell protein C receptor (PROCR) and annexin A2 (ANXA2)) to reduce cardiac xenograft rejection, and methods and materials for monitoring the progression, if any, of xenotransplant immunologic rejection.

### 2. Background Information

There is a chronic shortage of organs for transplantation. This is particularly the case in cardiac transplantation where approximately 2300 heart transplants are performed annually but up to 50,000 patients in chronic heart failure could benefit from a transplant. Xenotransplantation (transplantation from one species to another) could provide an unlimited supply of organs if successful. Xenotransplantation can be limited by an immunological rejection of the transplanted organ. Initially this rejection can be due to preformed antibodies present in humans and Old World primates that bind to a carbohydrate modification called the α-Gal antigen. This antigen can be produced in great abundance in pigs and other mammalian species. The combination of abundant α-Gal antigen in pig organs and high levels of preformed anti-Gal antibody in nonhuman primates (a model for humans) can result in a devastating hyperacute rejection of the graft usually within hours.

### SUMMARY

This document provides methods and materials for reducing cardiac xenograft rejection. For example, this document provides methods and materials for preparing transgenic pigs expressing reduced or no endogenous Sd^{a} or SDa-like glycans produced from a porcine β1,4 N-acetyl-galactosaminyl transferase 2 (B4GALNT2) glycosyltransferase and reduced or no endogenous α-Gal antigens produced from the porcine α1-3 galactosyl transferase (GT) glycosyltransferase, methods and materials for modifying a xenograft recipient's immunological response to non-Gal antigens (e.g., CD46, CD59, CD9, PROCR and ANXA2) to reduce cardiac xenograft rejection, and methods and materials for monitoring the progression of, if any, xenotransplant immunologic rejection. In some cases, this document provides methods for implanting a pig xenograft heart into a human. The pig xenograft donor can be a pig that contains genetic disruptions in α1-3 galactosyl transferase (GT) nucleic acid and β1,4 N-acetyl-galactosaminyl transferase 2 (B4GALNT2) nucleic acid. Such pigs can lack the ability to express Sd^{a} or SDa-like glycans and α-Gal antigens. The methods and materials described herein can be used to reduce immunogenicity of the pig to primate cardiac xenograft upon implantation and prolong the durability of the xenograft. This can benefit patients in chronic heart failure on the heart transplant waiting list for a donor heart.

In general, one aspect of this document features a method of providing a primate with a cardiac xenograft. The method includes implanting the cardiac xenograft into the primate, wherein the xenograft has decreased or no expression of α-Gal antigen and decreased or no expression of Sd^{a} or SDa-like antigen on the endothelial cell membranes.

In another embodiment, this document features a method of providing decreasing immune rejection of a cardiac xenograft. The method includes inducing antigen specific tolerance in a primate recipient wherein the antigen is at least one polypeptide selected from the group consisting of CD46, CD59, CD9, porcine PROCR and ANXA2.

In another embodiment, this document features a method for measuring the progress of cardiac xenograft immune rejection. The method includes monitoring a primate recipient antibody response to an individual non-Gal endothelial cell membrane antigen present on the xenograft, wherein the non-Gal endothelial cell membrane antigen is selected from the group consisting of CD46, CD59, CD9, porcine PROCR and ANXA2.

Unless otherwise defined, all technical and scientific terms used herein have the same meaning as commonly understood by one of ordinary skill in the art to which this invention pertains. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages of the invention will be apparent from the following detailed description, and from the claims.

### DESCRIPTION OF DRAWINGS

FIGS. 1A-B. Bar graphs depicting the non-Gal antibody levels in sensitized baboon serum used to identify non-Gal antigens. Non-Gal antibody responses were determined using flow cytometry to measure the extent of IgG binding to α-Gal antigen knock-out (GTKO) porcine aortic endothelial cells (PAECs). A. Reproduction of FIG. 1D from Davila et al. Xenotransplantation. 13(1):31-40 (2006) comparing baseline (Base) and necropsy IgG binding of three GT-positive cardiac xenograft recipients. B. Comparison of pretransplant (white bars) and necropsy (black bars) IgG binding to GTKO PAECs from a GTKO graft recipient. All recipients show an increase in anti-pig non-Gal IgG after transplant.

FIGS. 2A-D. Expression library screening and analysis. A. The map of the pRETRO-Lib vector used to clone the cDNA library from GT+ and GTKO porcine aortic endothelial cell mRNA. B. Human Embryonic Kidney 293 (HEK) cells infected with the pRETRO-PAEC viral library were stained with sensitized xenograft recipient sera. The brightest 10-30 percent of the cells were collected by cell sorting and regrown for additional rounds of enrichment. The filled distribution is antibody binding to the original infected population. The thin line is the population of selected cells restained with sensitized serum after 48 hours of growth. For each library screen, 3-7 rounds of enrichment were performed after which individual cells were sorted to 96-well plates, expanded and individually analyzed for IgG binding. C. Flow cytometry of individual pRETRO-PAEC library infected clones illustrating a variety of different levels of antibody binding. Filled histogram is a negative control that does not bind IgG. Thin lines represent IgG binding to individual clones. D. Library cDNA is recovered from genomic DNA of individual clones using PCR primers that flank the multiple cloning site (MCS) of the vector.

FIG. 3 is a table listing the results of BLAST searches of the NCBI GenBank database with the cDNA insert nucleic acid sequence for each of the six individual clones identified in the library screening.

FIGS. 4A-F. Initial identification of pRETRO-PAEC infected HEK cells expressing non-Gal PAEC antigens. The thin line represents sensitized baboon serum binding to individual pRETRO-PAEC library infected HEK cells isolated by flow cytometry and cell sorting. The background (filled histogram) is sensitized baboon serum staining uninfected HEK cells or HEK cells infected with an unrelated retrovirus. Cell lines expressing A. porcine CD9, B. porcine ANXA2, C. porcine B4GALNT2 D. porcine CD46, E. porcine CD59 and F. porcine PROCR have been identified.

FIG. 5A-L. Analysis of HEK cell lines transformed with pcDNA3.1 vector (Invitrogen) containing individual non-Gal antigen cDNA from pRETRO-PAEC infected isolates. Each stable cell line was screened with sensitized sera. A-F: Flow cytometry profiles of stable cell lines expressing A. porcine CD9, B. porcine PROCR, C. porcine CD46, D. porcine CD59, D; E. porcine ANXA2 and F. porcine B4GALNT2. G-L: Reverse transcriptase PCR analysis for RNA expression of E. porcine CD9, F. porcine PROCR, G. porcine CD46, H. porcine CD59, K. porcine ANXA2 and L. porcine B4GALNT2. Lanes 1-7 are RT-PCR products amplified from lane 1. pig heart, lane 2. PAEC, lanes 3-5. independent HEK transformants containing the nucleotide sequence for each respective non-Gal antigen, lane 6. untransformed HEK cells, lane 7. Flow cytometry negative HEK transformant for each respective non-Gal antigen, and lanes 8-10 are the same as lanes 3-5, however no RT-PCR was performed. These negative controls show that the products in lanes 3-5 are derived from RNA and not genomic DNA.

FIG. 6. Alignment of pig (SEQ ID NO:1), human (SEQ ID NO:2), and mouse (SEQ ID NO:3) B4GALNT2 amino acid sequences. A * indicates amino acid identity. Conservation is highest in the C-terminal region. Shaded regions highlight the conservation of the relationship between the exon boundaries of the genomic DNA, which are shaded dark and light, in the amino acid sequences of the protein for human and mouse. Boldfaced italic amino acids (325-339) indicated in the region encoded by exon 9 correspond to a conserved sequence detected in all three species of B4GALNT2 and detected in the human GM2 synthase. This sequence includes an acidic “DXD” motif conserved in 13 glycosyltransferase families of the CAZy classification. The DXD motif is required for GM2 enzymatic activity.

FIGS. 7A-E. Analysis of fluorescein isothiocyanate (FITC) conjugated Dolichos biflorus lectin (DBA) binding. A.DBA binding to HEK cells (filled) and HEK cells infected with a pRETRO virus expressing porcine B4GALNT2 (line). B. DBA binding to baboon aortic endothelial cells (thin line). The filled histogram is unstained baboon endothelial cells. C. DBA binding to GTKO porcine aortic endothelial cells (line). The filled histogram is unstained porcine endothelial cells. D. and E. show FITC-DBA staining of porcine and baboon heart tissue, respectively.

FIG. 8. The porcine cDNA (SEQ ID NO:4) and amino acid (SEQ ID NO:1) sequences. Vertical lines indicate the putative locations of exon boundaries based on the human and murine B4GALNT2 genes. The conserved amino acid sequence associated with the catalytic site of the enzyme is underlined. Boldface nucleotides indicate the position of PCR primers used to amplify intervening intron sequences from genomic porcine DNA. Arrows indicate the orientation of the primer for PCR.

FIG. 9. Genomic porcine nucleotide sequence (SEQ ID NO:5) for putative intron 8. Porcine genomic DNA sequences were amplified using primers from the B4GALNT2 cDNA corresponding to exons 8 and 9 in the conserved human and murine B4GALNT2 genes. Coding sequences from the porcine B4GALNT2 cDNA are presented in upper case and underlined. Boldface nucleotides indicate the positions of PCR primers used for amplification. Lowercase nucleotides represent intron sequence.

FIGS. 10A-B. Genomic porcine nucleotide sequence (SEQ ID NO:6) for putative intron 10. Porcine genomic DNA sequences were amplified using primers from the B4GALNT2 cDNA corresponding to exons 10 and 11 in the conserved human and murine B4GALNT2 genes. Coding sequences from the porcine B4GALNT2 cDNA are presented in upper case and underlined. Boldface nucleotides indicate the positions of PCR primers used for amplification. Lowercase nucleotides represent intron sequence. FIG. 10B. Top panel is a schematic diagram of the genomic organization of the pig B4GALNT2 gene. Bottom panel contains the exon-intron junction sequences of the pig B4GALNT2 gene. Sequence identifiers are in parentheses.

FIGS. 11A-C. Targeting vector for disruption of endogenous porcine B4GALNT nucleotide sequence. Illustration of the A. targeting vector, B. putative genomic structure of the porcine B4GALNT2 gene and C. resulting product produced after homologous recombination. Hash marks between A and B indicate regions of homologous recombination which result in the structure depicted in C. DTA is a polymerase 2 regulated diptheria toxin for negative selection. PGK-neomycin is a PGK regulated neomycin selectable marker. Numbers refer to putative exon structure of the porcine B4GALNT2 gene. These numbers are based on homology to the human and murine genes.

FIGS. 12A-L. Using non-Gal antigen expressing HEK cell lines to detect induced non-Gal antibody. Screening non-Gal immune responses using HEK transformed cells expressing non-Gal antigens. A cDNA for each of the non-Gal antigens in FIG. 3 was cloned into the mammalian expression vector pcDNA3.1/V5-His-TOPO. HEK cell lines expressing each of these cDNAs were incubated with pretransplant and necropsy serum (diluted 1:40) from a non-immunosuppressed heterotopic cardiac xenograft recipient and antibody binding was detected using a FITC conjugated anti-human IgG. Flow cytometry was used to determine the antibody response to each of the antigens. Panels A-F are pretransplant serum. Panels G-L are necropsy serum. The antigen expressed by the HEK cell line is listed above the pretransplant/necropsy serum pairs (A. and G. B4GALNT2, B. and H. PROCR, C. and I. CD9, D. and J. CD46, E. and K. CD59, F. and L. ANXA2). Specific staining is the thin line. Background staining is IgG binding to a G418 resistant HEK cell line transfected with the pcDNA3.1/V5-His-TOPO vector lacking an insert.

FIGS. 13A-H. Monitoring antibody responses to the Sd^{a}-like antigen response encoded by porcine B4GALNT2 expressed by the HEK-B4GALNT2 cell line. A-D. pretransplant and E-H. post explant serum reactivity to the HEK-B4GALNT2 cell line of GTKO heterotopic cardiac xenograft recipients. IgM (A and E) and IgG (B and F) reactivity is shown for a recipient that rejected the GTKO cardiac xenograft on day 27. IgM (C and G) and IgG (D and H) reactivity is shown for a recipient that rejected the GTKO cardiac xenograft on day 22. Background staining (filled) is serum reactivity to a G418 resistant HEK cell line transfected with pcDNA3.1/V5-His-TOPO vector without an insert. The transplants were performed under full immunosuppression as described in Byrne et al., Xenotransplantation, 15:268-276 (2008).

FIG. 14. Flow cytometry analysis of pre-transplant and sensitized necropsy serum IgG binding to HEK-B4GALNT2 cells. Each panel shows IgG binding to HEK-B4GALNT2 cells without absorption (labeled “g”) after repeated absorption with HEK cells labeled “r”) and after further repeated absorption with HEK-B4GALNT2 cells labeled “b”). The IgG binding present in necropsy serum is specific for a glycan encoded by porcine B4GALNT2, and uniquely present on HEK-B4GALNT2 cells, as absorption with HEK cells did not substantially affect IgG binding (compare “g” and “r” histograms)), but absorption with HEK-B4GALNT2 cells reduced IgG binding to back ground levels (“b” histogram). For absorption of the serum the pretransplant and necropsy serum samples (diluted 1:10) were incubated with 10⁷ HEK cells at 4° C. for 60 minutes. After incubation the cells were removed by centrifugation and the serum recovered. Each serum sample was absorbed three times. A portion of the HEK absorbed serum was then subsequently absorbed three times against 10⁷ HEK-B4GALNT2 cells. Background staining (filled histogram) is binding of the secondary PE-conjugated anti-human IgG only.

FIGS. 15A-B. Identification of potential non-Gal antibody oligosaccharide targets present on HEK-B4GALNT2 cells. A. Comparison of pretransplant (open) and necropsy (filled) IgG reactivity to four glycans present on the Mammalian printed glycan array (Version 4.2). Increased reactivity to these glycans is evident in necropsy serum. B. Structure of the oligosaccharides. Numbers correspond to values in the graphs. C. Shows the percent reduction in IgG binding after absorption of necropsy serum with HEK-B4GALNT2 cells. Abbreviations: Gal; galactose, GalNAc; N-Acetylgalactosamine, Glc; glucose, Neu5Ac; N-Acetylneuraminic acid, Man; mannose, GlcNAc; N-Acetylglucosamine

### DETAILED DESCRIPTION

This document provides methods and materials for reducing cardiac xenograft rejection. Provided herein are methods and materials for identifying pig heart non-Gal endothelial cell membrane antigens. Also provided are methods and materials for making transgenic pigs having disruptions in the endogenous β1,4 N-acetyl-galactosaminyl transferase 2 (B4GALNT2) nucleic acid sequence. Such transgenic pigs can be breed with transgenic pigs having disruptions in the endogenous GT nucleic acid sequence in order to produce transgenic pigs having disruptions in both nucleic acid sequences. Also provided are methods and materials for inducing immunological tolerance in a primate to pig heart endothelial cell membrane antigens (e.g. CD46, CD59, CD9 and porcine PROCR) prior to xenograft implantation and methods and materials for monitoring cardiac xenograft rejection.

### Identifying Pig Heart Non-Gal Endothelial Cell Membrane Antigens

This document provides methods and materials to identify non-Gal antigen targets of pig-to-primate cardiac xenograft immune rejection. As used herein, “non-Gal” refers to antigens different from the galactose α 1,3 galactose β 1,4N-acetylglucosamine trisaccharide (Gal α1-3Galβ1-4GlcNac; i.e., the α-Gal antigen).

An antibody response to the endothelium of the xenograft is widely considered to be the primary point of the immune response which initiates delayed xenograft rejection. Xenograft rejection is believed to occur due to chronic activation of the vascular endothelium of the graft by antibody binding or injury to the vascular endothelium through antibody directed cell cytotoxicity or complement mediated damage. These processes would promote the formation of a thrombogenic vasculature, resulting in microvascular thrombosis that, if unchecked, would lead to ischemic injury, culminating in coagulative necrosis of the myocardium. Prior to the development of GTKO pigs, rejection was thought to be induced primarily through the effects of anti-Gal antibody. The development of pigs deficient in the expression of the α-Gal antigen eliminated a role for anti-Gal antibody and revealed the significance of non-Gal antibody responses (Byrne et al., Xenotransplantation, 15:268-276 (2008)).

Any suitable method can be used to detect cardiac antigens that bind to antibodies from primate recipients of pig GT-positive and pig GTKO donor hearts. Examples include, but are not limited to, mammalian cDNA expression libraries screened with sensitized serum and sorted by flow cytometry, two-dimensional Western blot analysis, high throughput screening and proteomic analysis. Methods to identify such detected polypeptides include, but are not limited to mass spectrometry, nucleotide sequencing, amino acid sequencing and high performance liquid chromatography.

Preparing β1,4 N-acetyl-galactosaminyl transferase 2 and α1-3 galactosyl transferase knock-out pigs

This document provides transgenic pigs whose genomes have disruptions in the endogenous B4GALNT2 and GT nucleotide sequences. The human and mouse B4GALNT2 enzyme catalyzes the addition of N-acetylgalactosamine to terminal α2,3-sialylated galactose residues in the β1,4 linkage to produce the Sd^{a} antigen. This enzymatic activity has been detected in several species including the pig. The porcine B4GALNT2 gene identified herein is homologous to the human and murine genes and is expected to have similar enzymatic activity. The GT enzyme catalyzes the synthesis of galactose α 1,3 galactose B 1,4N-acetylglucosamine trisaccharide (the α-Gal antigen). The α-Gal antigen is found in most mammals, including pigs, but not in Old World monkeys, apes or humans.

Transgenic pigs whose genomes have disruptions in the endogenous porcine B4GALNT2 and GT nucleotide sequences can have reduced or no detectable porcine B4GALNT2 activity and reduced or no detectable GT activity. Cells from such transgenic pigs can have reduced or no detectable expression of the Sd^{a} or SDa-like glycans and α-Gal antigens on their surfaces. Such reduced or undetectable Sd^{a} or SDa-like glycans and α-Gal glycan expression is relative to control, non-transgenic pigs. For example, transgenic pigs having disruptions in the endogenous porcine B4GALNT2 and GT nucleotide sequences can present at least 50 percent less Sd^{a} and α-Gal antigen (e.g. less than 40 percent, less than 25 percent, less than 10 percent or less than 3 percent expression) as compared to control, non-transgenic pigs.

The term “endogenous” as used herein in reference to nucleic acid sequences and an organism refers to any nucleic acid sequence that is naturally present in the genome of that organism. An endogenous nucleic acid sequence can comprise one or more gene sequences, intergenic sequences, portions of gene sequences or intergenic sequences, or combinations thereof. The terms “B4GALNT2 nucleic acid sequence” and “GT nucleic acid sequence” as used herein, refer to the entire procine B4GALNT2 and GT gene sequences, including introns, exons, and regulatory regions.

Any suitable method can be used to generate pigs whose genomes contain disruptions in the endogenous B4GALNT2 and GT nucleic acid sequences. For example, transgenic porcine cells can be used for nuclear transplantation. Transgenic cells can be produced by introducing a knock-out construct into wild-type porcine cells. As used herein, a “knock-out construct” refers to a nucleic acid construct that is designed to disrupt an endogenous nucleic acid sequence (i.e., an endogenous porcine B4GALNT2 nucleic acid sequence or an endogenous GT nucleic acid sequence). Transgenic pigs whose genomes contain a disruption only in the GT nucleic acid sequence can be obtained commercially or can be produced as described elsewhere (see, e.g., Nottle et al., Xenotransplantation, 14(4): 339-344 (2007). The methods and materials provided herein can be used to design a disruption in a porcine endogenous B4GALNT2 nucleic acid sequence. A disruption can be positioned at many sites in the endogenous porcine B4GALNT2 nucleic acid sequence. Examples of disruptions include, but are not limited to, deletions in the native gene sequence and insertions of heterologous nucleic acid sequences into the native gene sequence. Examples of insertions can include, but are not limited to, artificial splice acceptors coupled to stop codons or splice donors coupled to fusion partners such as GFP. A knock-out construct can contain sequences that are homologous to the endogenous B4GALNT2 nucleic acid sequence or to sequences that are adjacent to the endogenous B4GALNT2 nucleic acid sequence. In some cases, a knock-out construct can contain a nucleic acid sequence encoding a selection marker (e.g., antibiotic resistance, a fluorescent reporter (e.g., GFP or YFP), or an enzyme (e.g., β-galactosidase)) operatively linked to a regulatory sequence (e.g., a promoter). A knock-out construct can include other nucleic acid sequences such as recombination sequences (e.g., loxP sequences, see Sendai, et al., Transplantation, 81(5):760-766 (2006)), splice acceptor sequences, splice donor sequences, transcription start sequences, and transcription stop sequences. Disruptions in the endogenous B4GALNT2 nucleic acid sequence can result in reduced expression of the gene or non-functional truncations or fusions of the encoded polypeptide.

Transgenic cells having a disruption in the endogenous B4GALNT2 nucleic acid sequence can be either adult or fetal cells and can be from primary or established cell lines. For example, transgenic fetal porcine fibroblasts can be fused with enucleated oocytes. Fused, activated oocytes can be cultured to the blastocyst stage, and implanted into a recipient. See, Arat, et al., Biol. Reprod., 66(6):1768-1774 (2002); and DeBoer, et al., U.S. Pat. No. 5,633,076. Adult somatic cells of any cell type including, for example, granulosa cells and fibroblast cells, also can be used to produce transgenic pigs (Arat, et al., MoI. Reprod. Dev., 60(1):20-26 (2001); and Arat, et al., (2002), supra, respectively). Nuclei can be removed from genetically modified adult somatic cells, and transplanted into enucleated oocytes. After activation, the eggs can be cultured to the 2-8 cell stage, or to the blastocyst stage, and implanted into a suitable recipient (DeBoer, et al., supra). Transgenic pigs heterozygous for the disrupted B4GALNT2 gene can be mated to produce homozygous transgenic pigs.

Transgenic pigs can be identified using any appropriate method. For example, cells from animals obtained using nuclear transplantation can be assessed for endogenous B4GALNT2 nucleic acid sequence disruption, B4GALNT2 RNA expression, or B4GALNT2 polypeptide expression. For example, endogenous B4GALNT2 nucleic acid sequence disruption can be identified using methods including southern blotting and PCR. B4GALNT2 RNA expression can be determined using methods such as Northern blot analysis, RT-PCR and fluorescent in situ hybridization. B4GALNT2 polypeptide expression can be determined using methods such as western blot analysis, immunohistochemistry, immunofluorescence, and detecting expression of the Sd^{a} antigen on tissue sections. The methods for identifying transgenic pigs listed are intended to provide examples and are not in any way meant to limit the scope of the invention.

To determine if the B4GALNT2 antigen is present on the surface of cells from heterozygous or homozygous transgenic animals, tissue can be removed from the animal and then embedded using, for example, OCT (TISSUE-TEK, Sakura) embedding medium. Tissues can be sectioned, placed on glass slides, air-dried, and stored at −80° C. until use. The sectioned tissues can be stained for the Sd^{a} antigen after fixing the sections in acetone, washing in water, blocking the slides, then incubating with the Dolichos biflorus (DBA) lectin. DBA is commercially available (e.g., from United States Biological (Swampscott, MA)). DBA can be labeled. Suitable labels include, without limitation, radionuclides (e.g., 125I, 1311, 35S, 3H, 32P, 33P, or 14C), fluorescent moieties (e.g., fluorescein, PerCP, rhodamine, or phycoery thrin), luminescent moieties (e.g., QDot Nanoparticles from Quantum Dot Corporation, Palo Alto, CA), or enzymes (e.g., alkaline phosphatase or horseradish peroxidase). DBA can be directly or indirectly labeled. Methods of indirect labeling can include, for example, conjugating the DBA with biotin then contacting the DBA-biotin with avidin or streptavidin labeled with a molecule described above. Methods of detecting or quantifying a label depend on the nature of the label and are known in the art. Examples of detectors include, without limitation, x-ray film, radioactivity counters, scintillation counters, spectrophotometers, colorimeters, fluorometers, luminometers, and densitometers. Combinations of these approaches (including “multi-layer” assays) familiar to those in the art can be used to enhance the sensitivity of assays.

Transgenic pigs whose genomes have disruptions in both the endogenous B4GALNT2 and GT nucleotide sequences can be obtained by breeding. Crossing a pig that has little or no Sd^{a} antigen expression due to a disruption in the B4GALNT2 nucleotide sequence with a pig that has little or no α-Gal antigen expression due to a disruption in the GT nucleotide sequence can be performed to produce transgenic pigs with disruptions in both B4GALNT2 and GT nucleotide sequences. It can be determined if the offspring of the mating contain disruptions in both the endogenous B4GALNT2 and GT nucleotide sequences by RT-PCR, Northern blot analysis, nucleotide sequencing, immunoblot analysis, PCR, Southern blot analysis, flow cytometry with DBA lectin and other methods known in the art.

### Modifying the Xenograft Recipient's Immunological Response to Non-Gal Antigens

The term “tolerance” as used herein refers to the specific immunological unresponsiveness to an antigen resulting from a previous exposure to such antigen. Antigen specific tolerance can be induced in a mammal (for example, mouse, rat, rabbit, dog, pig, goat, cow, Old World primate, human, etc.) by any mechanism known in the art. For example, molecular chimerism can be used to induce antigen specific tolerance in a recipient nonhuman primate. A retroviral or lentiviral vector encoding non-Gal antigens can be used to transduce recipient bone marrow derived hematopoietic stem cells. Such cells can be reintroduced to the nonhuman primate recipient prior to xenotransplantation. These transduced cells will travel to the immune compartments, establish a level of molecular chimerism and express the non-Gal antigens in the context of “self” without generating inflammatory co-signals. This will modulate the immune response to these non-Gal antigens as they will now be perceived as “self.”

As another example, antigen specific tolerance can also be induced by ex vivo exposure of immune cells from a nonhuman primate recipient to an alloantigen (e.g. non-Gal antigen). Dendritic cells (DCs) act to present foreign antigens to T lymphocytes. When this presentation occurs under inflammatory conditions where both the antigen and secondary co-stimulating signals are present, DCs induce T cell activation. In the absence of inflammation or co-stimulatory signals, DC presentation of antigen to T cell will induce a state of tolerance either through T cell deletion, anergy or the expansion of antigen specific T regulatory cells. Immature DCs are isolated from the nonhuman primate recipient prior to transplantation and exposed to purified non-Gal antigens (e.g. CD46, CD59, CD9 and porcine PROCR), cells expressing the non-Gal antigens (e.g. HEK cells or any mammalian cell line that does not express α-Gal), or exosomes from apoptotic cells that express the non-Gal antigens. The antigen pulsed DCs are then returned to the recipient prior to transplantation. The effectiveness of the procedure prior to and after xenotransplantation can be monitored using standard T cell proliferation assays where recipient T cells are stimulated by non-Gal antigens or cells expressing the non-Gal antigens.

### Measuring the Progress of Pig Heart Xenograft Rejection

The polypeptide non-Gal antigens (i.e. CD46, CD59, CD9 and porcine PROCR) can be expressed in any mammalian cell line that does not express α-Gal (i.e. human, ape and Old World primate cell lines). For example, the polypeptide non-Gal antigens can be expressed in Human Embryonic Kidney 293 cells (ATCC, (Manassas, VA)). While mammalian cells were used to express recombinant polypeptide non-Gal antigens, bacteria, yeast or insect cells can be used to produce the recombinant non-Gal antigens. These recombinant polypeptides can include the entire amino acid sequence or can be limited to the extracellular domain or some other subset of the amino acid sequence involved in binding to the non-Gal antibody. In some cases, the polypeptide non-Gal antigen cDNAs can be expressed as fusion proteins. These may include, but are not limited to, various polypeptide tags (i.e. 6x histidine tags, Flag tags, in vivo biotinylation sequences, myc tags, the immunoglobin constant region and other commonly used sequences designed to assist purification of recombinant proteins). These polypeptide tags can be located at either the amino or carboxyl terminus of the recombinant non-Gal antigens.

The recombinant non-Gal antigens can be bound to solid substrates and used to establish assays for monitoring non-Gal antigen immune responses. For example, the recombinant proteins can be bound to ELISA plates or spotted onto paper or glass supports. These substrates can be used to assay the presence of non-Gal antibody using standard ELISA and protein array methods known in the art. Recombinant non-Gal antigen can also be attached to flow cytometry beads and antibody binding to these beads determined using fluorochrome conjugated anti-human IgG or IgM which cross reacts with nonhuman primate IgG or IgM. Peptide sequences from the non-Gal antigens that bind preformed or induced non-Gal antibody can also be used as substrate for monitoring non-Gal antigen immune responses.

The invention will be further described in the following examples, which do not limit the scope of the invention described in the claims.

### EXAMPLES

### Example 1—Identifying Non-Gal Antigens Present on the Endothelial Cell Membranes of Pig Hearts Involved in Immunologic Xenograft Rejection

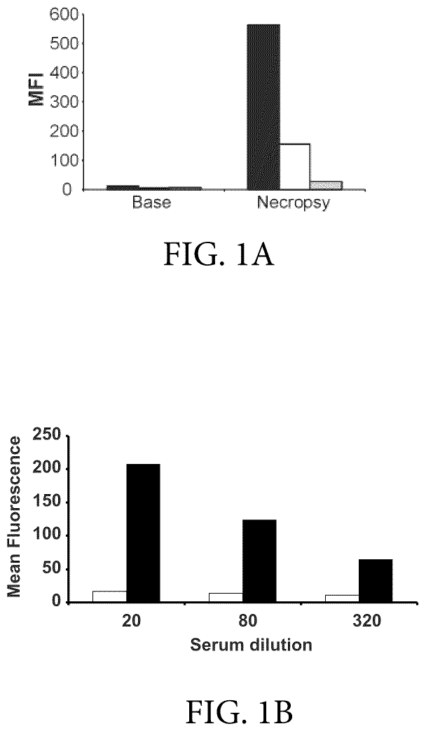

Heterotopic pig-to-primate cardiac xenografts were performed using GT-positive and GTKO donor hearts without immunosuppression. See Davila et al., Xenotransplantation. 13(1):31-40 (2006). Sera Obtained at Necropsy was Screened by flow cytometry to measure IgG binding to GTKO porcine aortic endothelial cells (PAECs). Serum from both GT-positive and GTKO recipients showed an induced antibody response to non-Gal antigens as evidenced by increased IgG binding to GTKO PAECs in necropsy sera compared to pre-transplant sera (FIG. 1). This necropsy sera was used for the library screen.

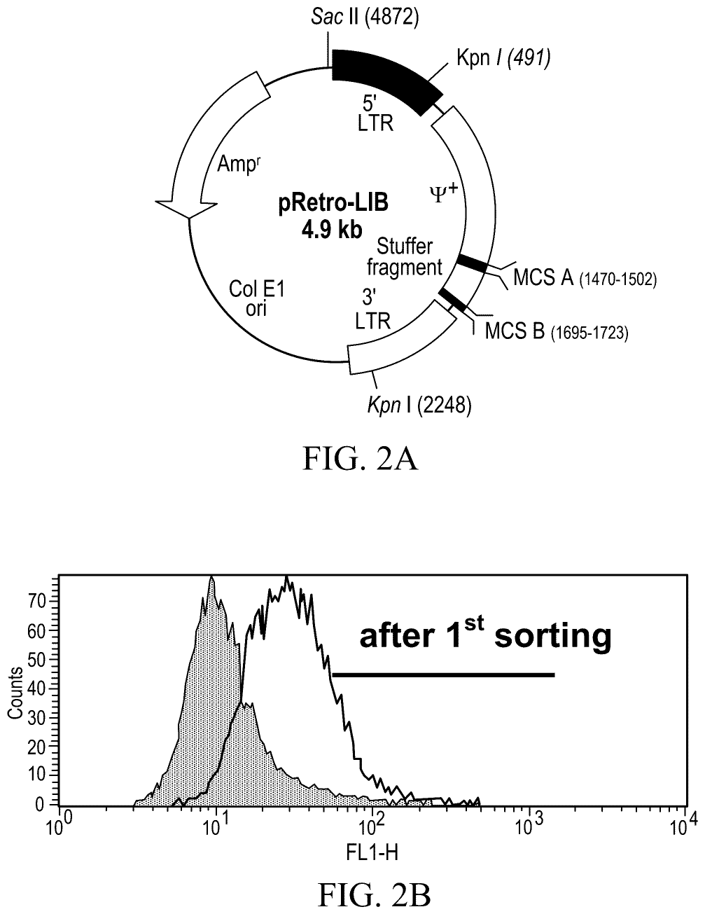

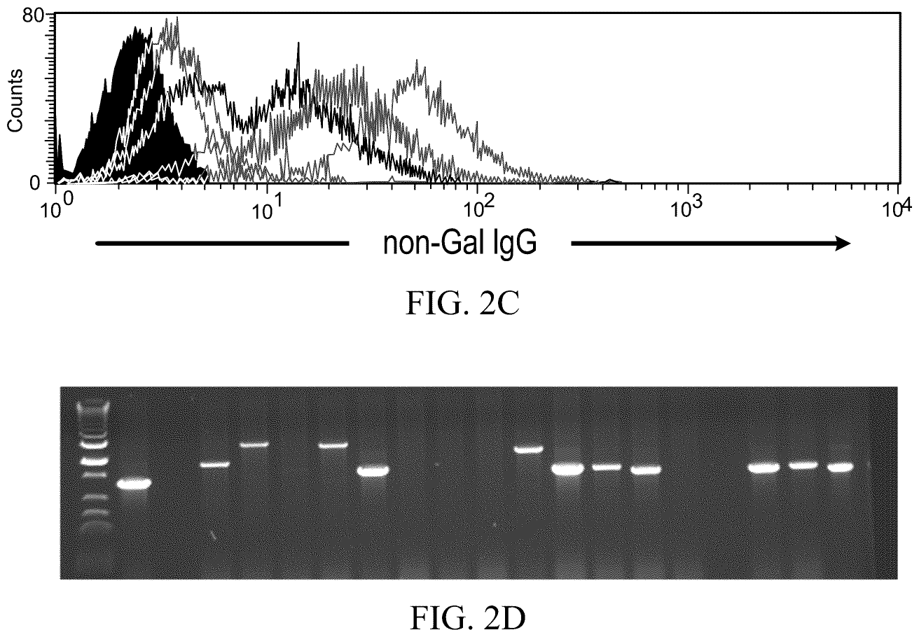

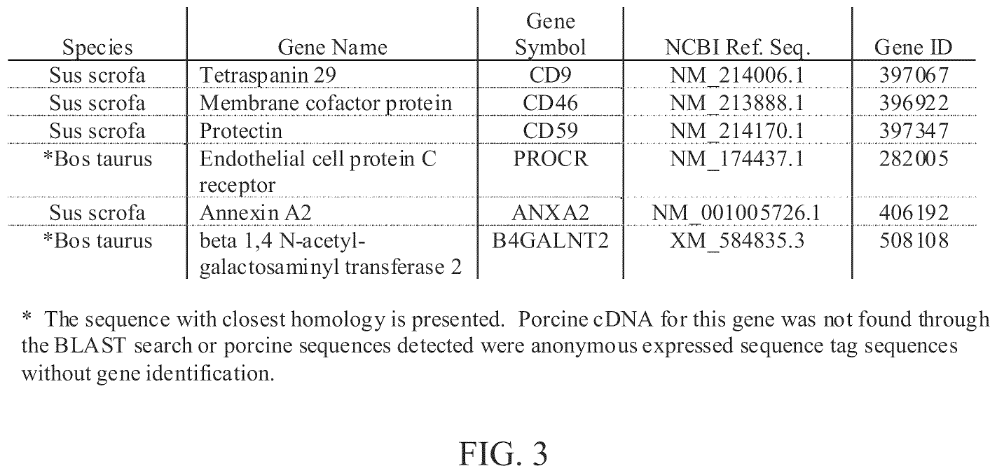

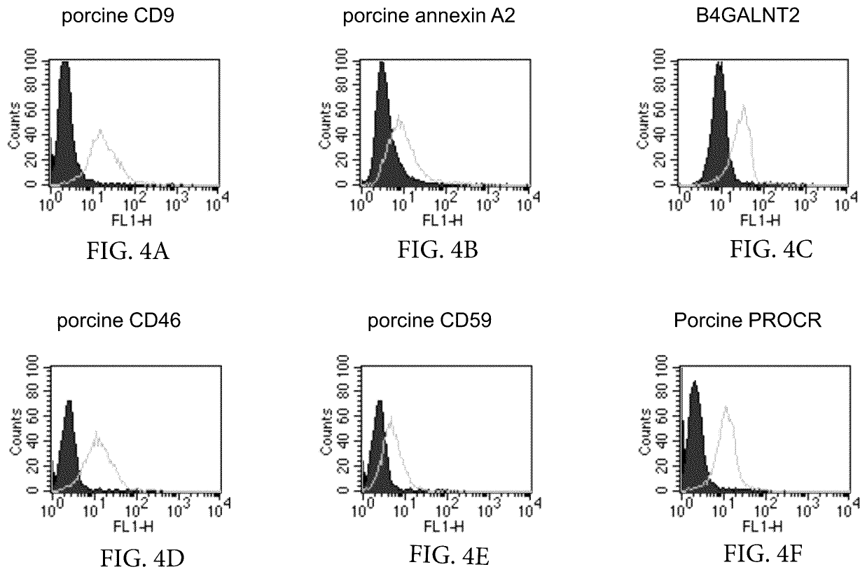

A standard cDNA expression library was produced using mRNA from GT-positive and GTKO porcine aortic endothelial cells. This library (pRETRO-PAEC) was made in the pRetro-LIB vector (Clontech, FIG. 2A) and was co-transfected with pVSV-G (encoding an envelope glycoprotein from the vesicular stomatitis virus) into GP2-293 packaging cells to produce a pantropic high titer retroviral library stock. The pRETRO-PAEC viral stock was used to infect HEK cells, which were stained with sensitized serum from a pig-to-primate cardiac xenograft recipient 48 hours after infection and then sorted by flow cytometry to collect the brightest 10-30 percent of the cells (FIG. 2B). These bright cells were regrown for 48-72 hours before being selected again. A total of 3-7 rounds of selection were performed, to enrich the population with cells that bound sensitized IgG, prior to isolation of individual cells by flow cytometry. Individual clones were then rescreened for antibody binding (FIG. 2C) and the cDNA insert from antibody positive clones was recovered by PCR amplification of genomic DNA (FIG. 2D) using primers that flank the multiple cloning site of the vector. The amplified product was cloned into a TA-cloning vector (Invitrogen) for sequencing. The sequence of the cDNA insert was used for a BLAST search of the NCBI GenBank database to identify the encoded gene. FIG. 3 lists the results of that BLAST search, showing the species of origin, the common gene names, gene symbol, NCBI reference identification and gene identification number for each of six non-Gal proteins identified in the library screen. For four of these non-Gal polypeptides (CD46, CD59, CD9 and ANXA2) the corresponding pig gene was already identified. In two instances the pig gene had not been previously reported so a bovine homologue, which showed the highest degree of homology, is listed (PROCR and B4GALNT2). A summary of the flow cytometry profile for each of these non-Gal expressing pRETRO-PAEC infected HEK cell lines is presented in FIG. 4.

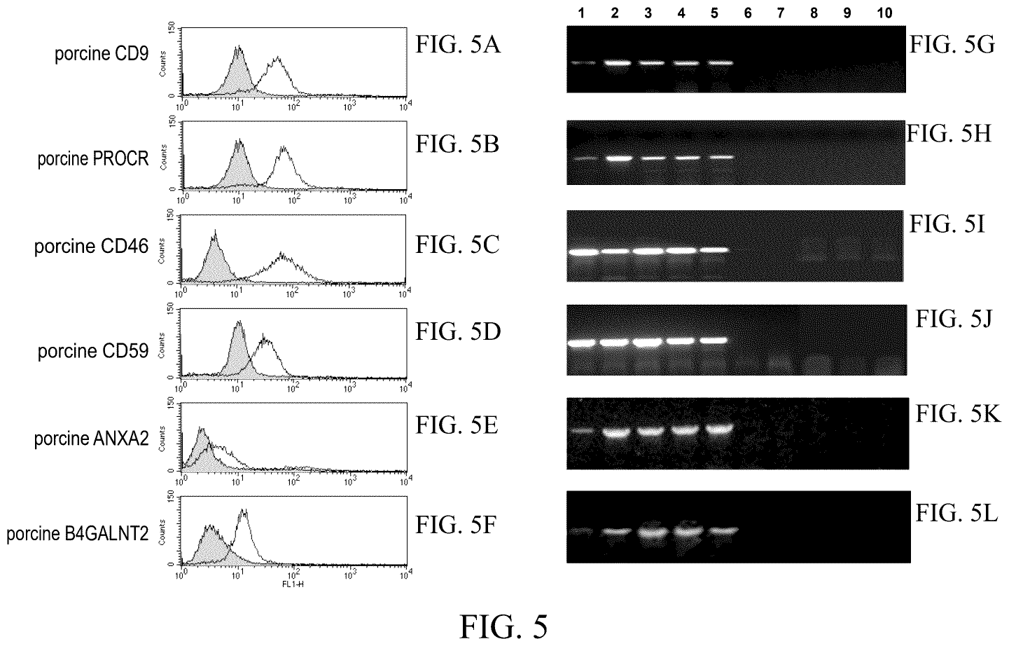

The pRETRO-PAEC infected HEK cells have a possibility of being infected with more than one virus and may express more than one porcine cDNA. To insure that the identified non-Gal target antigens are the authentic targets of the induced antibody response, each of the non-Gal cDNAs was individually cloned into pcDNA3.1/V5-His-TOPO (Clontech) and transfected into HEK cells. These HEK transformants were selected for G418 resistance and a stable cell line for each non-Gal antigen was produced. These stable cell lines were rescreened with sensitized sera to validate that an induced antibody response was directed towards each of the non-Gal antigens (FIG. 5A-F). Further, RNA samples from porcine hearts and cultured endothelial cells were used to confirm expression of the non-Gal antigens in the transformed HEK cells (FIG. 5G-L) by reverse transcriptase PCR using non-Gal antigen nucleotide sequence specific primers. Antibody to most of the polypeptide non-Gal antigens identified can initiate delayed xenograft rejection through antibody directed complement mediated injury to the vascular endothelial cells but can also exacerbate delayed xenograft rejection by blocking the normal functions of these proteins (CD46, CD59, CD9, porcine PROCR and ANXA2). Both CD46 and CD59 are known as complement regulatory proteins (CRPs). These membrane proteins are widely expressed and normally function to limit complement mediated damage to autologous cells. Because they are membrane proteins, they act locally on the cell surface and establish an intrinsic barrier to complement mediated damage. They do not systemically deplete complement. CD46 is a cofactor for complement factor I and functions to cleave surface bound C3b and C4b to block a key amplification step in the complement cascade and CD59 blocks the polymerization of the membrane attack complex, the terminal step in complement activation which leads to cell lysis. In xenotransplantation, the use of human CRP transgenes (CD46, CD55 and CD59) has been central to endowing donor organs with greater resistance to complement mediated damage. It was initially believed that human and porcine CRPs acted in an essentially species specific fashion, with the CRPs of each species being effective only against complement of the autologous species. While some aspect of this may be true, it is also clear that enhanced expression of CRP function, even porcine CRPs, will act to limit primate complement induced damage. Antibody responses to these porcine CRPs may block the function of these proteins and thereby effectively reduce the overall complement regulatory capacity of the organ. Induced antibody to porcine CRPs would place the donor organ at greater risk for antibody directed complement mediated damage.

The polypeptide non-Gal antigen CD9 is a tetraspanin protein family member. These proteins have four hydrophobic transmembrane domains. CD9 has two extracellular protein loops where most of the amino acid variation between species is found. CD9 is well known for its presence on platelets and anti-CD9 antibodies efficiently activate platelets that in some instances can induce a lethal thrombosis. CD9 is also expressed on endothelial cells where it forms tetraspanin enriched microdomains (TEM). Antibodies to CD9 on endothelial cells promote neutrophil adhesion, possibly through endothelial cell activation or by cross linking TEMs to aggregate neutrophil adhesion proteins VCAM and ICAM. In the context of a xenograft, an antibody response to porcine CD9 (on endothelium) promotes neutrophil adhesion and subsequent endothelial cell activation or injury. If the induced anti-CD9 cross reacts with recipient CD9, even to a limited extent, then the antibody might effectively cross link recipient platelets to the endothelium of the xenograft creating a potent thrombogenic effect.

The non-Gal antigen PROCR acts on the endothelial cell surface to enhance the formation of activated protein C by the complex of thrombin and thrombomodulin. Activated protein C is a prominent anticoagulant due to its cleavage of coagulation factors VI and VIIIa which reduce thrombin generation. PROCR can also be shed from the endothelial cell surface by the effects of metalloproteinase. The soluble receptor-activated protein C complex appears to bind to neutrophils and decrease their binding to the endothelium. Anti-inflammatory effects have also been associated with PROCR-activated protein C complex. Antibodies have been isolated which block the function of PROCR (Ye et al., Biochem Biophys Res Commun. 259(3):671-677 (1999)). Antibody with this specificity in a xenograft recipient can enhance coagulation and xenograft rejection.

ANXA2 was identified as a porcine non-Gal endothelial cell membrane antigen (Byrne et al., Xenotransplantation, 15:268-276 (2008)). Annexins are a family of diverse genes which encode proteins with calcium regulated phospholipid and membrane binding functions. The annexins are mainly considered intracellular proteins that act as anchors connecting cytoskeletal elements to the membrane and supporting membrane-membrane interactions. As such, they are implicated in exocytosis, endocytosis and stabilization of organelle and plasma membrane domains. Some annexins, including the identified non-Gal antigen ANXA2, are found on the extracellular surface and have extracellular functions. ANXA2 is an endothelial cell surface receptor for plasminogen and tissue type plasminogen activator (tPA). Consistent with this, ANXA2 knock-out mice show reduced levels of tPA dependent plasmin generation and exhibit incomplete clearance of arterial thrombi (Cockrell et al., Lupus. 17(10):943-951 (2008)). It may be anticipated that extracellular ANXA2 can promote fibrinolysis and may thereby forestall graft rejection by limiting the extent of thrombosis. Anti-ANXA2 antibodies that block the proposed ANXA2 fibrinolytic function could in effect promote thrombosis within the xenograft. Additionally, antibody responses to ANXA2 have been detected in patients with antiphospholipid syndrome and shown to cause endothelial cell activation and the induction of tissue factor which would also contribute to a prothrombotic vasculature (Cesarman-Maus et al., Blood. 107(11):4375-4382 (2006)).

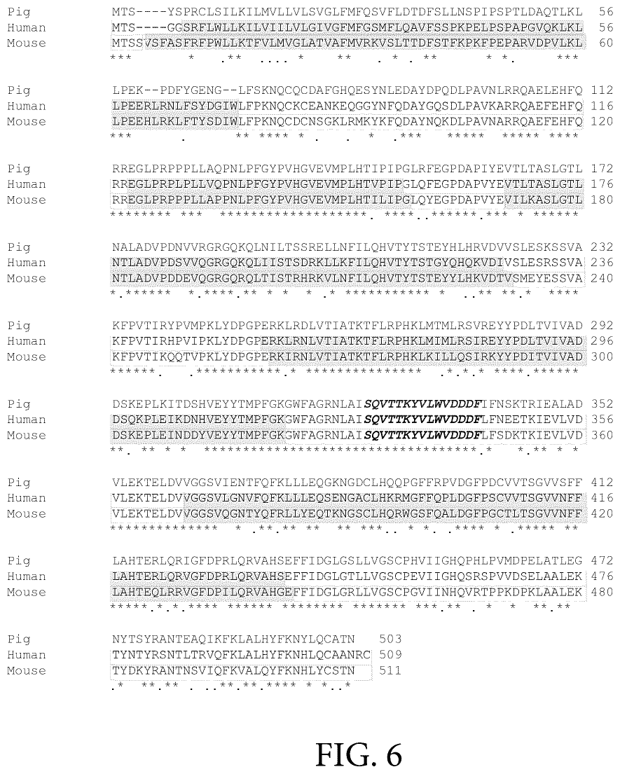

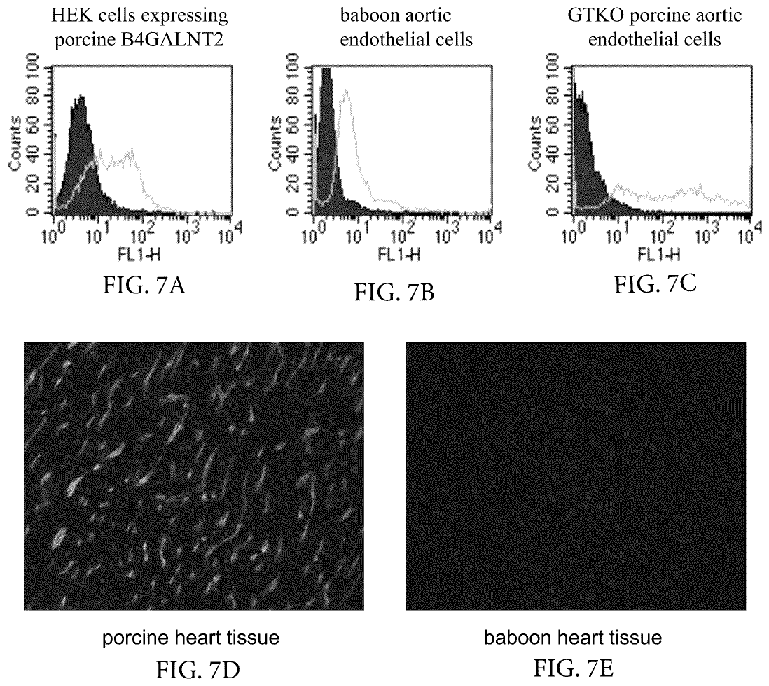

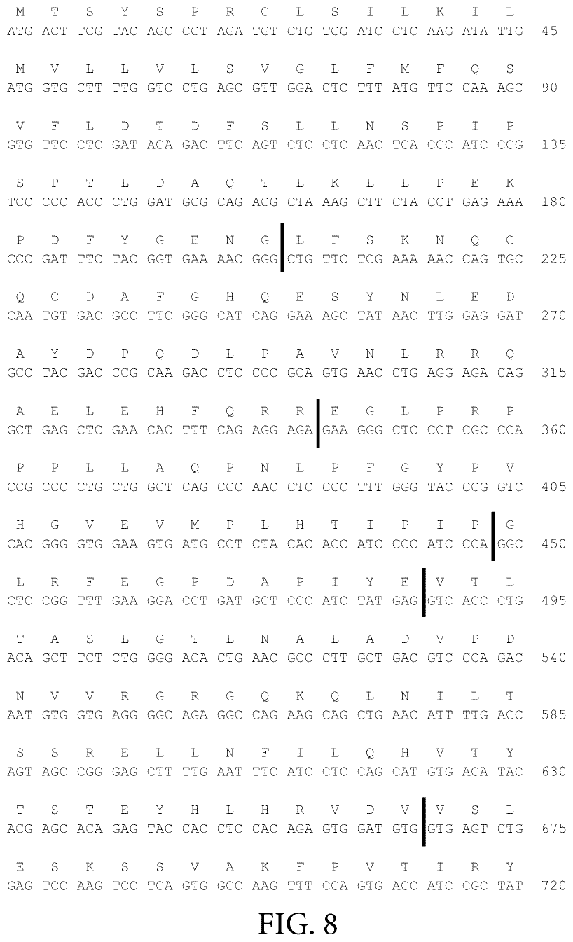

The B4GALNT2 enzyme was also identified as a non-Gal antigen in the library screen. The gene for this enzyme has been cloned from humans and mice. The enzyme catalyzes the addition of N-acetylgalactosamine to terminal α2,3-sialylated galactose residues in the β1,4 linkage to produce the Sd^{a} antigen. There is a 74 percent and 68 percent amino acid identity between the translated porcine polypeptide sequence isolated herein and the human and murine B4GALNT2 polypeptides respectively (FIG. 6). The porcine B4GALNT2 gene is expected to encode a polypeptide with similar, though not necessarily identical, enzymatic activity as seen for the human and murine sequences. The Sd^{a} antigen, also known as CAD, is expressed on 90 percent of Caucasian red blood cells, in human urine as the Tamm-Horsfall glycoprotein, in the oxyntic mucosa of the stomach as a glycolipid and in the colonic mucosa as a glycoprotein. The Dolichos biflorus lectin (DBA) can be used to detect the Sd^{a} antigen by binding to the β-linked N-acetylgalactosamine (Kamada et al., J Biochem, 109(1):178-183 (1991); Piller et al., European journal of biochemistry/FEBS, 191(2):461-466 (1990). The DBA lectin binds poorly to HEK cells but shows increased binding to HEK cells infected with the pRETRO vector expressing porcine B4GALNT2 (FIG. 7A). There is a high level of DBA binding to porcine aortic endothelial cells but minimal binding to baboon aortic endothelial cells (FIGS. 7B and C). Likewise, lectin staining of porcine and baboon heart samples show a high level of DBA binding to porcine endothelium with little if any binding to baboon heart (FIGS. 7D and E). This is consistent with previous DBA staining of porcine femoral arteries and microvascular endothelial cells (Solanes et al., Anatomia, histologia, embryologia, 34(2):105-111 (2005); Johnson et al., Microvascular research. 64(2):278-288 (2002)). Alloantibodies to the Sd^{a} antigen have been noted at low frequency in military veterans and an induced response to Sd^{a} has been observed in some patients after transfusion (Tormey et al., Transfusion, 48(10):2069-2076 (2008); Spitalnik et al., Vox Sang. 42(6):308-312 (1982)). The identification of the B4GALNT2 cDNA nucleotide sequence in the library screen suggests that an anti-Sd^{a} or antibody response to an Sd^{a}-like carbohydrate on the xenograft may be present in non-human primates.

### Example 2—Engineering a Targeted Disruption in the Porcine B4GALNT2 Nucleotide Sequence

The cDNA sequence of porcine B4GALNT2 and its conservation in human and mouse provides the needed information to design a targeting vector suitable for disrupting the porcine B4GALNT2 gene using the standard methods of homologous recombination. The amino acids encoded by the nucleotides of individual exons in the human and murine genes show conservation (FIG. 6). This is a common observation in mammalian genes when exons from different species encode the same or similar portions of a polypeptide. This conservation suggests that the porcine B4GALNT2 gene is made up of 11 coding exons and suggests the approximate location for exon boundaries within the porcine cDNA.

The porcine, human and murine B4GALNT2 cDNA sequences exhibit a high level of conservation in the region that encodes the C-terminal portion of the polypeptide. This region of the human B4GALNT2 cDNA sequence encodes a portion of the B4GALNT2 polypeptide that is important for enzymatic activity (Montiel et al., Biochem J., 373:369-379 (2003)). This is likely to be similar in the porcine B4GALNT2 polypeptide based on conservation. This region is also conserved by the related human GM2 synthase that encodes an N-acetylgalactosamine transferase polypeptide. Human GM2 and human, murine and porcine B4GALNT2 share a conserved amino acid sequence (SQVTTKYVLWVDDDF (SEQ ID NO:7)) encoded by exon 9 (boldface in FIG. 6). Within this sequence is an acidic DXD motif (underlined) that is conserved in 13 glycosyltransferase families of the CAZy classification and is required for GM2 enzymatic activity (Li et al., Glycobiology, 11:217-229 (2001)). This suggests that the amino acids encoded by exon 9 in the human and murine B4GALNT nucleotide sequences are involved in the catalytic site and that the corresponding porcine amino acid sequence will have a similar function. The putative exons 9 and 10 of the porcine B4GALNT2 nucleotide sequence were deleted in the construct. The loss of such sequences, that include the conserved sequence associated with the enzymatic active site, should effectively eliminate the function of the gene.

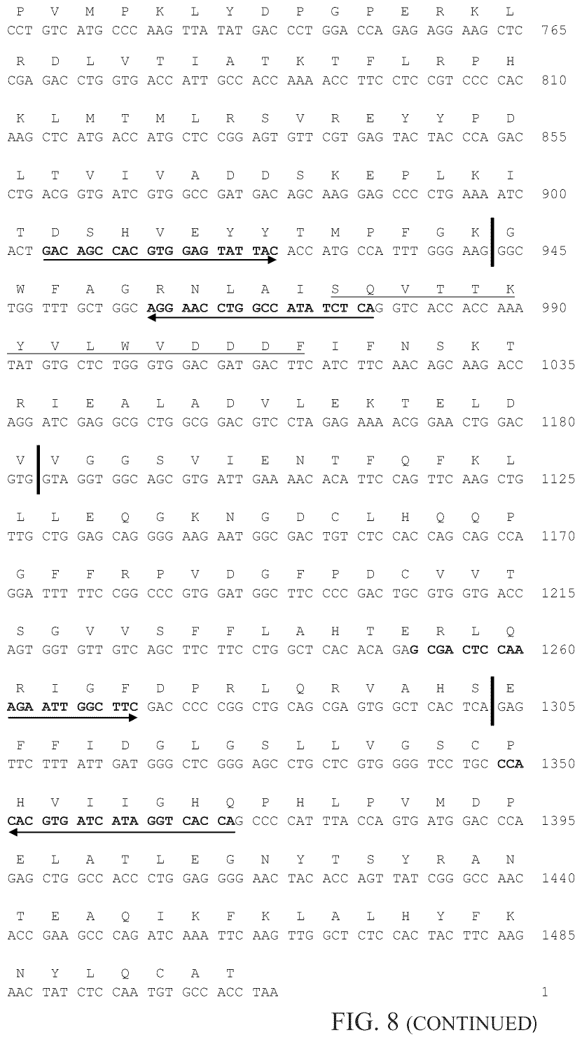

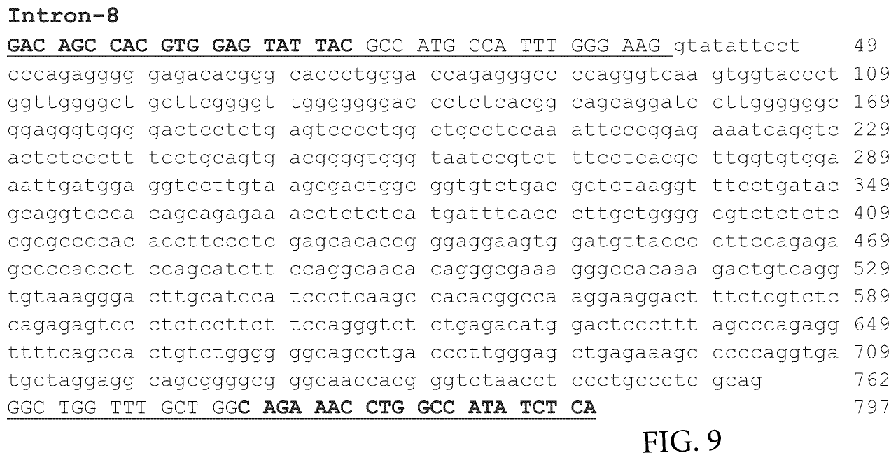

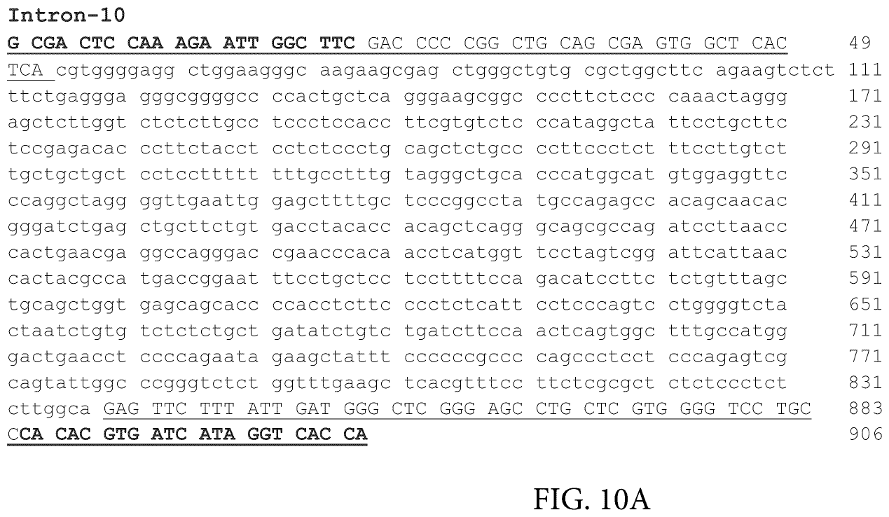

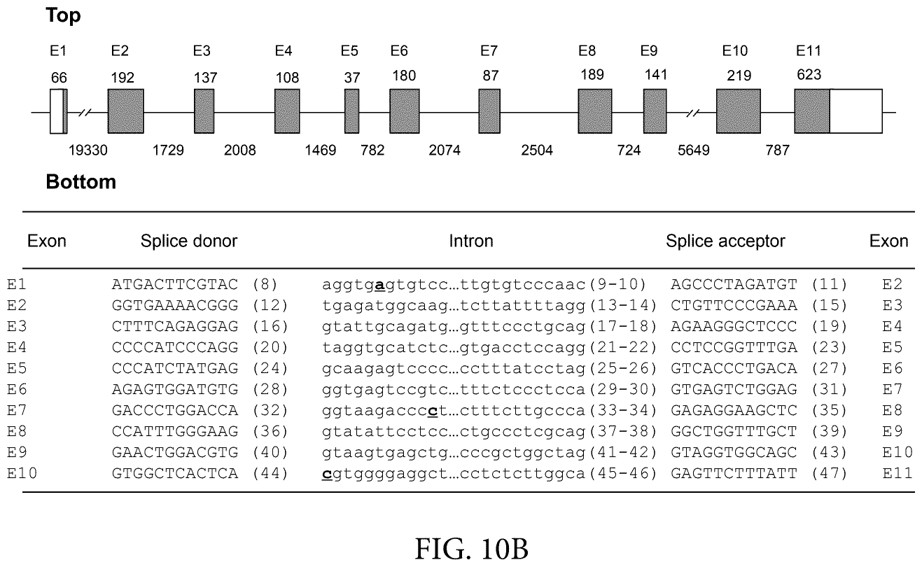

To design a targeting vector suitable for disrupting the porcine B4GALNT2 gene requires 200-1000 basepairs of homologous porcine sequences that flank the targeted neomycin insertion site. These sequences cannot come directly from the B4GALNT2 cDNA as mammalian genes are composed of a series of highly dispersed exons which are spliced together to produce the cDNA. Instead genomic DNA that immediately flanks the desired insertion site must be used. Based on the conserved exon encoded portions of the B4GALNT2 protein in humans and mice, the porcine gene likely consists of 11 coding exons encoding approximately 5, 63, 46, 35, 13, 60, 29, 63, 47, 73, and 69 amino acids each in that order (FIG. 8). To isolate genomic DNA flanking the enzymatic active site we designed PCR primers (FIG. 8), based on the conserved exon protein relationships exhibited by human and murine B4GALNT. These primers amplify the porcine genomic DNA between the presumed exons 8 and 9 and exons 10 and 11 of the porcine B4GALNT gene (FIG. 8). The porcine genomic PCR products were sequenced (FIGS. 9 and 10). Each shows the known primer sequence, and an adjacent 14-47 nucleotides of porcine B4GALNT2 coding sequence. This confirms that the PCR products are derived from the porcine B4GALNT gene and that the intervening sequences represent authentic intron sequences of the porcine B4GALNT2 gene. These introns would correspond to the homologous introns 8 and 10 based on the structure of the human and mouse genes. This strategy has been repeated for all of the intron and exon boundaries for the porcine B4GALNT2 gene, confirming the presence of 11 coding exons and providing essential noncoding genomic sequences for isolating any combination of intron and exons (FIG. 10B).

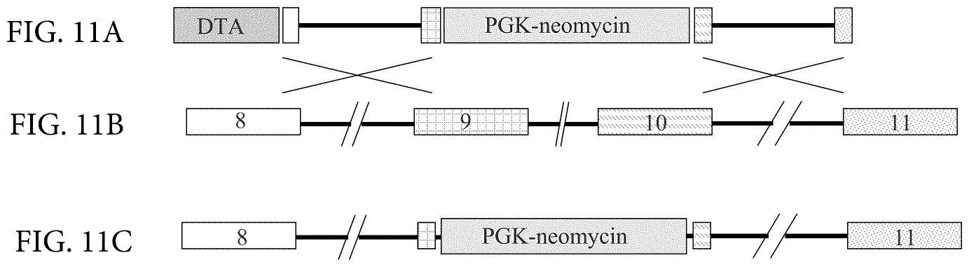

The intron 8 and intron 10 PCR products provide the homologous genomic sequences needed to produce a targeting vector to disrupt the B4GALNT2 gene in a manner analogous to the process used to disrupt the GGTA-1 locus (Sharma et al., Transplantation, 75:430-436 (2003). This vector would consist of the following components; a polymerase 2 regulated diphtheria toxin A gene (DTA), 5′ flanking homologous sequences including portions of the porcine B4GALNT2 coding sequences and the intron 8 sequence, a PGK-neomycin resistance cassette and 3′ flanking homologous sequences including portions of the porcine B4GALNT2 coding sequence and the intron 10 sequence (FIG. 11). The DTA gene provides a negative selectable marker to minimize the frequency of non-homologous insertions. Homologous recombination events within the 5′ and 3′ flanking sequences result in a loss of the DTA and an insertion of the PGK-Neo marker. This PGK-Neo insertion effectively deletes most of the coding sequence encoded by putative exons 9 and 10 of the porcine B4GALNT2 gene. The loss of these sequences, which would include the conserved sequence associated with the enzymatic active site and the insertion of the PGK-Neo gene would effectively eliminate the function of the gene. The construct could further include frame shift alterations and in frame termination codons to further insure disruption of the B4GALNT2 gene.

### Example 3—Monitoring the Progress of Pig Heart Xenograft Rejection

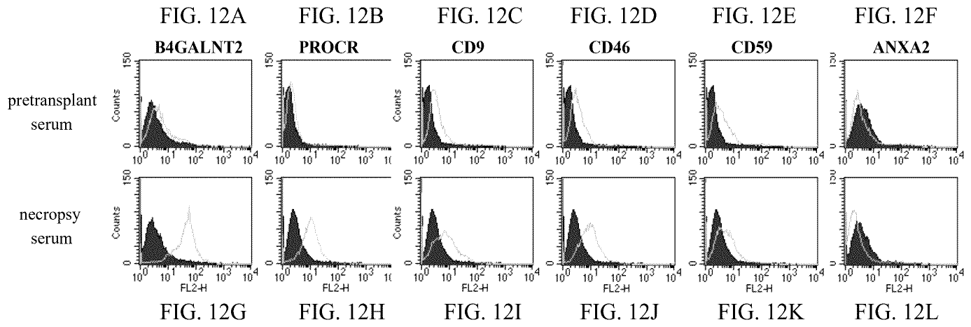

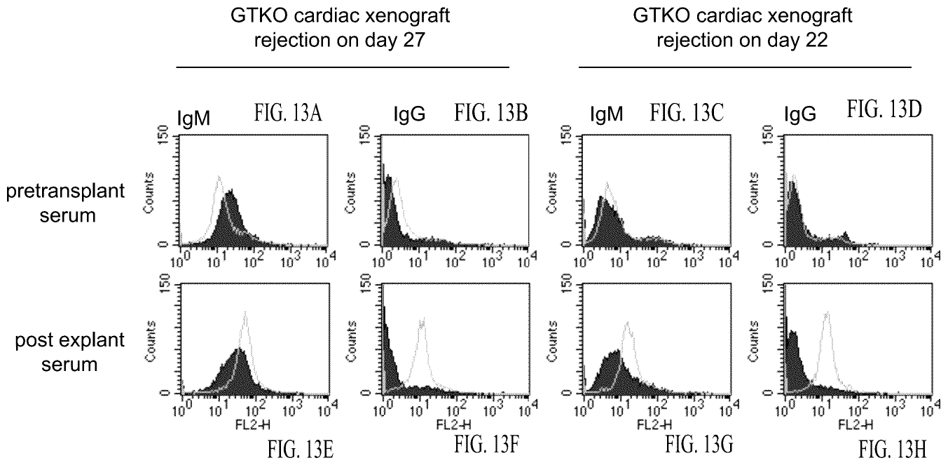

Utilizing cDNAs encoding the polypeptide non-Gal antigens identified herein, HEK cell lines were developed expressing each of the polypeptide non-Gal antigens with the exception of B4GALNT2. These cell lines were directly used to monitor non-Gal antibody responses to individual non-Gal antigens (i.e. CD46, CD59, CD9, PROCR and ANXA2). Stable HEK cell lines expressing each of these cDNAs were incubated with pre-transplant and necropsy serum (diluted 1:40) from a non-immunosuppressed heterotopic cardiac xenograft recipient. Antibody binding was detected using a FITC conjugated anti-human IgG. Flow cytometry was used to determine the antibody response to each of the antigens (FIG. 12). The xenograft recipient had a strongly induced antibody response to the B4GALNT2 antigen and PROCR, a less intense though positive response to porcine CD9 and CD46 and a minimal response to porcine CD59 and ANXA2. This same strategy can be used for any porcine xenograft recipient and is not limited to non-immunosuppressed recipients. FIG. 13 illustrates the induced antibody response to the B4GALNT2 antigen in two GTKO pig to primate recipients which were fully immunosuppressed. In each case a weak induction of IgM and a stronger induction of B4GALNT2 reactive IgG was detected.

### Example 4—Glycan Array Analysis

The HEK-B4GALNT2 cell line, identified in a library screen, expresses a porcine glycosyltransferase similar to B4GALNT2 in humans and mice (FIG. 6). The murine and human B4GALNT2 transferase can add N-acetylgalactosamine to galactose containing an alpha 2, 3 sialic acid residue. This results in formation of the SDa glycan. In the HEK-B4GALNT2 cells, expression of the porcine B4GALNT2 gene leads to formation of a new non-Gal glycan, not present on normal HEK cells, which is the target of an induced antibody response after pig-to-primate xenotransplantation. The sequence homology shared between the porcine glycosyltransferase and the human and mouse B4GALNT2 genes (FIG. 6) suggests that the porcine gene is likely to perform a similar enzymatic function resulting in expression of an SDa or SDa-like glycan on the surface of the HEK-B4GALNT2 cells. This suggests that an SDa or SDa-like glycan can be a new non-Gal carbohydrate antigen, however, the precise enzymatic function of the porcine gene product is not known and variation in the protein sequence may alter its enzymatic activity compared to the human and murine genes. Furthermore, expression of porcine B4GALNT2 in HEK cells can compete with endogenous glycosyltransferases and may alter the glycan composition.

A mammalian printed glycan array (Version 4.2) produced by the Consortium of Functional Glycomics, directed by James Paulson, Department of Chemical Physiology, Scripps Institute, (http://www.functionalglycomics.org/static/consortium/organization.shtml) was used to measure the anti-glycan specificity present in post-transplant sensitized primate serum as a means of further defining the glycans present on HEK-B4GALNT2 cells. Glycan arrays are similar to those described elsewhere (Wong et al., Curr. Opin. Chem. Biol., 12:86-92 (2008) and Paulson et al., Nature Chem. Biol., 2(5):238-248 (2006)).

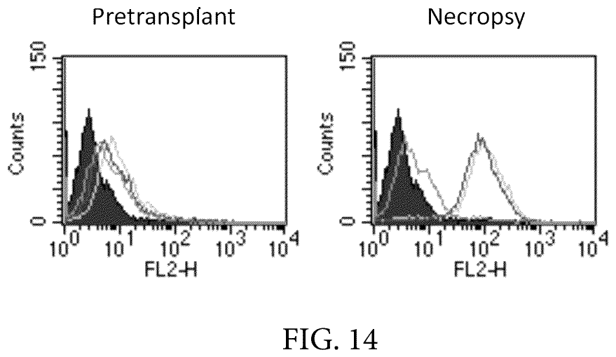

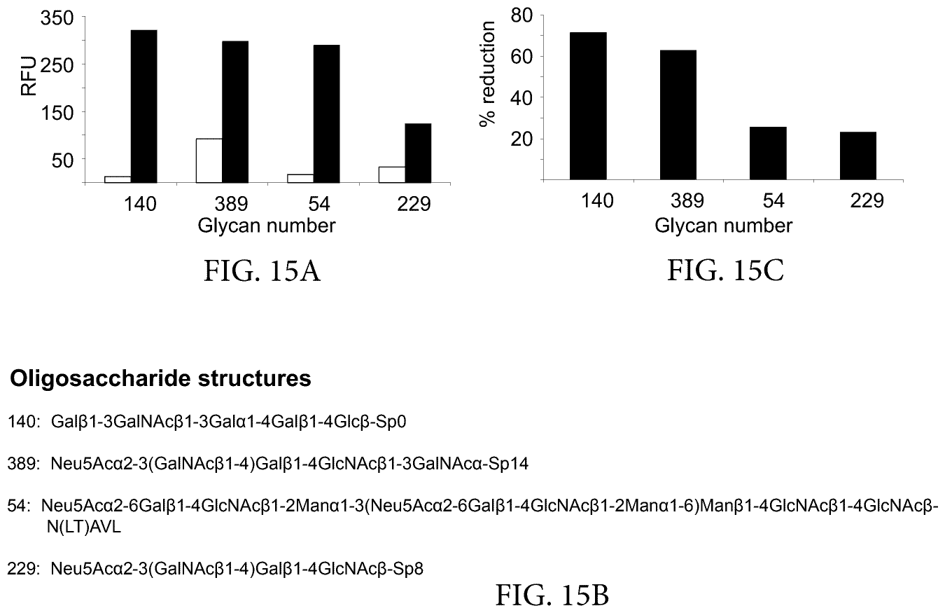

Pretransplant and sensitized necropsy serum from a GTKO pig-to-primate cardiac xenograft recipient contained, in addition to other non-Gal specificities, a high level of HEK-B4GALNT2 reactivity (FIG. 14). This reactivity was specific for the glycan produced by expression of porcine B4GALNT2 and uniquely present on HEK-B4GALNT2 cells as absorption of the sensitized serum with HEK cells did not diminish IgG binding to HEK-B4GALNT2 cells whereas absorption with HEK-B4GALNT2 cells reduced IgG binding to background levels. These serum sources were used to probe the mammalian glycan array to identify glycan structures with increased IgG binding in the sensitized necropsy serum (FIGS. 15A and B). The identified structures included the SDa antigen (structure 389) and oligosaccharides containing N-Acetylneuraminic acid substitutions or N-Acetylgalactosamine residues. These structures are broadly compatible with the presumed activity of the porcine B4GALNT2 gene and with the lectin DBA binding detected on porcine endothelial cells and on HEK-B4GALNT2 cells (FIG. 7). When necropsy serum was absorbed with HEK-B4GALNT2 cells, reactivity to these glycans was reduced (FIG. 15C) by 20-70%, suggesting that these, or similar structures, are present on the HEK-B4GALNT2 surface.

While the glycan array data were largely qualitative, the structures identified in FIG. 15 may be present on the surface of HEK-B4GALNT2 cells, but there may be additional related structures which were not detected in this analysis and may contribute to the reduction in IgG binding after absorption. Also the porcine B4GALNT2 enzyme may act directly to produce a certain structure, such as SDa, but it may also act indirectly, through competitive interaction with other glycosyltransferases present in HEK cells, to produce increased levels of other oligosaccharides which may also contribute to non-Gal IgG binding to these cells. For these reasons, this analysis is not an exhaustive enumeration of the oligosaccharides produced by porcine B4GALNT2 or oligosaccharides that may contribute to non-gal IgG binding to this cell line.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1-6. (canceled)

7. A method for measuring the progress of cardiac xenograft immune rejection, wherein said method comprises:
(a) obtaining a pre-implantation serum sample from a primate,
(b) implanting (i) pig cardiac tissue whose genome comprises a homozygous disruption of the endogenous β1,4 N-acetyl-galactosaminyl transferase 2 (B4GALNT2) gene into said primate, wherein said tissue does not express a functional B4GALNT2 comprising the amino acid sequence of SEQ ID NO:1, and does not express Sd^{a} glycans or SD^{a}-like glycans, (ii) pig cardiac tissue whose genome comprises a homozygous disruption of the endogenous α1-3 galactosyl transferase (GT) gene into said primate, wherein said tissue does not express a functional GT, and does not express α-Gal, or (iii) pig cardiac tissue whose genome comprises said homozygous disruption of the endogenous B4GALNT2 gene and said homozygous disruption of the endogenous GT gene into said primate, wherein said tissue does not express a functional B4GALNT2 comprising the amino acid sequence of SEQ ID NO: 1 or a functional GT, and does not express Sd^{a} glycans, SD^{a}-like glycans, or α-Gal,
(c) obtaining a post-implantation serum sample from said primate,
(d) delivering a cDNA encoding CD46, CD59, CD9, porcine protein C receptor (PROCR), annexin A2 (ANXA2), or said B4GALNT2 to a cell line, wherein said cell line expresses said CD46, CD59, CD9, PROCR, ANXA2, or B4GALNT2,
(e) contacting cells of said cell line with said pre-implantation serum sample and determining the level of IgM or IgG binding to said cells, thereby obtaining first binding results,
(f) contacting cells of said cell line with said post-implantation serum sample and determining the level of IgM or IgG binding to said cells, thereby obtaining second binding results, and
(g) detecting a higher level of IgM or IgG binding for said first binding results as compared to the level of IgM or IgG binding for said second binding results.


SEQUENCE LISTING (47 sequences)

SEQ ID NO: 1 - protein, 503 aa, Sus scrofa
MTSYSPRCLSILKILMVLLVLSVGLFMFQSVFLDTDFSLLNSPIPSPTLDAQTLKLLPEKPDFYGENGLFSKNQCQCDAFGHQESYNLEDAYDPQDLPAVNLRRQAELEHFQRREGLPRPPPLLAQPNLPFGYPVHGVEVMPLHTIPIPGLRFEGPDAPIYEVTLTASLGTLNALADVPDNVVRGRGQKQLNILTSSRELLNFILQHVTYTSTEYHLHRVDVVSLESKSSVAKFPVTIRYPVMPKLYDPGPERKLRDLVTIATKTFLRPHKLMTMLRSVREYYPDLTVIVADDSKEPLKITDSHVEYYTMPFGKGWFAGRNLAISQVTTKYVLWVDDDFIFNSKTRIEALADVLEKTELDVVGGSVIENTFQFKLLLEQGKNGDCLHQQPGFFRPVDGFPDCVVTSGVVSFFLAHTERLQRIGFDPRLQRVAHSEFFIDGLGSLLVGSCPHVIIGHQPHLPVMDPELATLEGNYTSYRANTEAQIKFKLALHYFKNYLQCATN

SEQ ID NO: 2 - protein, 509 aa, Homo sapiens
MTSGGSRFLWLLKILVIILVLGIVGFMFGSMFLQAVFSSPKPELPSPAPGVQKLKLLPEERLRNLFSYDGIWLFPKNQCKCEANKEQGGYNFQDAYGQSDLPAVKARRQAEFEHFQRREGLPRPLPLLVQPNLPFGYPVHGVEVMPLHTVPIPGLQFEGPDAPVYEVTLTASLGTLNTLADVPDSVVQGRGQKQLIISTSDRKLLKFILQHVTYTSTGYQHQKVDIVSLESRSSVAKFPVTIRHPVIPKLYDPGPERKLRNLVTIATKTFLRPHKLMIMLRSIREYYPDLTVIVADDSQKPLEIKDNHVEYYTMPFGKGWFAGRNLAISQVTTKYVLWVDDDFLFNEETKIEVLVDVLEKTELDVVGGSVLGNVFQFKLLLEQSENGACLHKRMGFFQPLDGFPSCVVTSGVVNFFLAHTERLQRVGFDPRLQRVAHSEFFIDGLGTLLVGSCPEVIIGHQSRSPVVDSELAALEKTYNTYRSNTLTRVQFKLALHYFKNHLQCAANRC

SEQ ID NO: 3 - protein, 511 aa, Mus musculus
MTSSVSFASFRFPWLLKTFVLMVGLATVAFMVRKVSLTTDFSTFKPKFPEPARVDPVLKLLPEEHLRKLFTYSDIWLFPKNQCDCNSGKLRMKYKFQDAYNQKDLPAVNARRQAEFEHFQRREGLPRPPPLLAPPNLPFGYPVHGVEVMPLHTILIPGLQYEGPDAPVYEVILKASLGTLNTLADVPDDEVQGRGQRQLTISTRHRKVLNFILQHVTYTSTEYYLHKVDTVSMEYESSVAKFPVTIKQQTVPKLYDPGPERKIRNLVTIATKTFLRPHKLKILLQSIRKYYPDITVIVADDSKEPLEINDDYVEYYTMPFGKGWFAGRNLAISQVTTKYVLWVDDDFLFSDKTKIEVLVDVLEKTELDVVGGSVQGNTYQFRLLYEQTKNGSCLHQRWGSFQALDGFPGCTLTSGVVNFFLAHTEQLRRVGFDPILQRVAHGEFFIDGLGRLLVGSCPGVIINHQVRTPPKDPKLAALEKTYDKYRANTNSVIQFKVALQYFKNHLYCSTN

SEQ ID NO: 4 - DNA, 1509 bp, Sus scrofa
  source 1..1509: mol_type = genomic DNA
atgacttcgtacagccctagatgtctgtcgatcctcaagatattgatggtgcttttggtcctgagcgttggactctttatgttccaaagcgtgttcctcgatacagacttcagtctcctcaactcacccatcccgtcccccaccctggatgcgcagacgctaaagcttctacctgagaaacccgatttctacggtgaaaacgggctgttctcgaaaaaccagtgccaatgtgacgccttcgggcatcaggaaagctataacttggaggatgcctacgacccgcaagacctccccgcagtgaacctgaggagacaggctgagctcgaacactttcagaggagagaagggctccctcgcccaccgcccctgctggctcagcccaacctcccctttgggtacccggtccacggggtggaagtgatgcctctacacaccatccccatcccaggcctccggtttgaaggacctgatgctcccatctatgaggtcaccctgacagcttctctggggacactgaacgcccttgctgacgtcccagacaatgtggtgaggggcagaggccagaagcagctgaacattttgaccagtagccgggagcttttgaatttcatcctccagcatgtgacatacacgagcacagagtaccacctccacagagtggatgtggtgagtctggagtccaagtcctcagtggccaagtttccagtgaccatccgctatcctgtcatgcccaagttatatgaccctggaccagagaggaagctccgagacctggtgaccattgccaccaaaaccttcctccgtccccacaagctcatgaccatgctccggagtgttcgtgagtactacccagacctgacggtgatcgtggccgatgacagcaaggagcccctgaaaatcactgacagccacgtggagtattacaccatgccatttgggaagggctggtttgctggcaggaacctggccatatctcaggtcaccaccaaatatgtgctctgggtggacgatgacttcatcttcaacagcaagaccaggatcgaggcgctggcggacgtcctagagaaaacggaactggacgtggtaggtggcagcgtgattgaaaacacattccagttcaagctgttgctggagcaggggaagaatggcgactgtctccaccagcagccaggatttttccggcccgtggatggcttccccgactgcgtggtgaccagtggtgttgtcagcttcttcctggctcacacagagcgactccaaagaattggcttcgacccccggctgcagcgagtggctcactcagagttctttattgatgggctcgggagcctgctcgtggggtcctgcccacacgtgatcataggtcaccagccccatttaccagtgatggacccagagctggccaccctggaggggaactacaccagttatcgggccaacaccgaagcccagatcaaattcaagttggctctccactacttcaagaactatctccaatgtgccacctaa

SEQ ID NO: 5 - DNA, 798 bp, Sus scrofa
  source 1..798: mol_type = genomic DNA
gacagccacgtggagtattacgccatgccatttgggaaggtatattcctcccagagggggagacacgggcaccctgggaccagagggccccagggtcaagtggtaccctggttggggctgcttcggggttgggggggaccctctcacggcagcaggatccttggggggcggagggtggggactcctctgagtcccctggctgcctccaaattcccggagaaatcaggtcactctccctttcctgcagtgacggggtgggtaatccgtctttcctcacgcttggtgtggaaattgatggaggtccttgtaagcgactggcggtgtctgacgctctaaggtttcctgatacgcaggtcccacagcagagaaacctctctcatgatttcacccttgctggggcgtctctctccgcgccccacaccttccctcgagcacaccgggaggaagtggatgttaccccttccagagagccccaccctccagcatcttccaggcaacacagggcgaaagggccacaaagactgtcaggtgtaaagggacttgcatccatccctcaagccacacggccaaggaaggactttctcgtctccagagagtccctctccttcttccagggtctctgagacatggactccctttagcccagaggttttcagccactgtctggggggcagcctgacccttgggagctgagaaagcccccaggtgatgctaggaggcagcggggcgggcaaccacgggtctaacctccctgccctcgcagggctggtttgctggcagaaacctggccatatctca

SEQ ID NO: 6 - DNA, 907 bp, Sus scrofa
  source 1..907: mol_type = genomic DNA
gcgactccaaagaattggcttcgacccccggctgcagcgagtggctcactcacgtggggaggctggaagggcaagaagcgagctgggctgtgcgctggcttcagaagtctctttctgagggagggcggggccccactgctcagggaagcggccccttctccccaaactagggagctcttggtctctcttgcctccctccaccttcgtgtctcccataggctattcctgcttctccgagacacccttctacctcctctccctgcagctctgccccttccctctttccttgtcttgctgctgctcctccttttttttgcctttgtagggctgcacccatggcatgtggaggttcccaggctaggggttgaattggagcttttgctcccggcctatgccagagccacagcaacacgggatctgagctgcttctgtgacctacaccacagctcagggcagcgccagatccttaacccactgaacgaggccagggaccgaacccacaacctcatggttcctagtcggattcattaaccactacgccatgaccggaatttcctgctcctccttttccagacatccttctctgtttagctgcagctggtgagcagcaccccacctcttcccctctcattcctcccagtcctggggtctactaatctgtgtctctctgctgatatctgtctgatcttccaactcagtggctttgccatgggactgaacctccccagaatagaagctatttccccccgccccagccctcctcccagagtcgcagtattggcccgggtctctggtttgaagctcacgtttccttctcgcgctctctccctctcttggcagagttctttattgatgggctcgggagcctgctcgtggggtcctgcccacacgtgatcataggtcacca

SEQ ID NO: 7 - protein, 15 aa, Homo sapiens
SQVTTKYVLWVDDDF

SEQ ID NO: 8 - DNA, 12 bp, Sus scrofa
  source 1..12: mol_type = genomic DNA
atgacttcgtac

SEQ ID NO: 9 - DNA, 12 bp, Sus scrofa
  source 1..12: mol_type = genomic DNA
aggtgagtgtcc

SEQ ID NO: 10 - DNA, 12 bp, Sus scrofa
  source 1..12: mol_type = genomic DNA
ttgtgtcccaac

SEQ ID NO: 11 - DNA, 12 bp, Sus scrofa
  source 1..12: mol_type = genomic DNA
agccctagatgt

SEQ ID NO: 12 - DNA, 12 bp, Sus scrofa
  source 1..12: mol_type = genomic DNA
ggtgaaaacggg

SEQ ID NO: 13 - DNA, 12 bp, Sus scrofa
  source 1..12: mol_type = genomic DNA
tgagatggcaag

SEQ ID NO: 14 - DNA, 12 bp, Sus scrofa
  source 1..12: mol_type = genomic DNA
tcttattttagg

SEQ ID NO: 15 - DNA, 12 bp, Sus scrofa
  source 1..12: mol_type = genomic DNA
ctgttcccgaaa

SEQ ID NO: 16 - DNA, 12 bp, Sus scrofa
  source 1..12: mol_type = genomic DNA
ctttcagaggag

SEQ ID NO: 17 - DNA, 12 bp, Sus scrofa
  source 1..12: mol_type = genomic DNA
gtattgcagatg

SEQ ID NO: 18 - DNA, 12 bp, Sus scrofa
  source 1..12: mol_type = genomic DNA
gtttccctgcag

SEQ ID NO: 19 - DNA, 12 bp, Sus scrofa
  source 1..12: mol_type = genomic DNA
agaagggctccc

SEQ ID NO: 20 - DNA, 12 bp, Sus scrofa
  source 1..12: mol_type = genomic DNA
ccccatcccagg

SEQ ID NO: 21 - DNA, 12 bp, Sus scrofa
  source 1..12: mol_type = genomic DNA
taggtgcatctc

SEQ ID NO: 22 - DNA, 12 bp, Sus scrofa
  source 1..12: mol_type = genomic DNA
gtgacctccagg

SEQ ID NO: 23 - DNA, 12 bp, Sus scrofa
  source 1..12: mol_type = genomic DNA
cctccggtttga

SEQ ID NO: 24 - DNA, 12 bp, Sus scrofa
  source 1..12: mol_type = genomic DNA
cccatctatgag

SEQ ID NO: 25 - DNA, 12 bp, Sus scrofa
  source 1..12: mol_type = genomic DNA
gcaagagtcccc

SEQ ID NO: 26 - DNA, 12 bp, Sus scrofa
  source 1..12: mol_type = genomic DNA
cctttatcctag

SEQ ID NO: 27 - DNA, 12 bp, Sus scrofa
  source 1..12: mol_type = genomic DNA
gtcaccctgaca

SEQ ID NO: 28 - DNA, 12 bp, Sus scrofa
  source 1..12: mol_type = genomic DNA
agagtggatgtg

SEQ ID NO: 29 - DNA, 12 bp, Sus scrofa
  source 1..12: mol_type = genomic DNA
ggtgagtccgtc

SEQ ID NO: 30 - DNA, 12 bp, Sus scrofa
  source 1..12: mol_type = genomic DNA
tttctccctcca

SEQ ID NO: 31 - DNA, 12 bp, Sus scrofa
  source 1..12: mol_type = genomic DNA
gtgagtctggag

SEQ ID NO: 32 - DNA, 12 bp, Sus scrofa
  source 1..12: mol_type = genomic DNA
gaccctggacca

SEQ ID NO: 33 - DNA, 12 bp, Sus scrofa
  source 1..12: mol_type = genomic DNA
ggtaagacccct

SEQ ID NO: 34 - DNA, 12 bp, Sus scrofa
  source 1..12: mol_type = genomic DNA
ctttcttgccca

SEQ ID NO: 35 - DNA, 12 bp, Sus scrofa
  source 1..12: mol_type = genomic DNA
gagaggaagctc

SEQ ID NO: 36 - DNA, 12 bp, Sus scrofa
  source 1..12: mol_type = genomic DNA
ccatttgggaag

SEQ ID NO: 37 - DNA, 12 bp, Sus scrofa
  source 1..12: mol_type = genomic DNA
gtatattcctcc

SEQ ID NO: 38 - DNA, 12 bp, Sus scrofa
  source 1..12: mol_type = genomic DNA
ctgccctcgcag

SEQ ID NO: 39 - DNA, 12 bp, Sus scrofa
  source 1..12: mol_type = genomic DNA
ggctggtttgct

SEQ ID NO: 40 - DNA, 12 bp, Sus scrofa
  source 1..12: mol_type = genomic DNA
gaactggacgtg

SEQ ID NO: 41 - DNA, 12 bp, Sus scrofa
  source 1..12: mol_type = genomic DNA
gtaagtgagctg

SEQ ID NO: 42 - DNA, 12 bp, Sus scrofa
  source 1..12: mol_type = genomic DNA
cccgctggctag

SEQ ID NO: 43 - DNA, 12 bp, Sus scrofa
  source 1..12: mol_type = genomic DNA
gtaggtggcagc

SEQ ID NO: 44 - DNA, 12 bp, Sus scrofa
  source 1..12: mol_type = genomic DNA
gtggctcactca

SEQ ID NO: 45 - DNA, 12 bp, Sus scrofa
  source 1..12: mol_type = genomic DNA
cgtggggaggct

SEQ ID NO: 46 - DNA, 12 bp, Sus scrofa
  source 1..12: mol_type = genomic DNA
cctctcttggca

SEQ ID NO: 47 - DNA, 12 bp, Sus scrofa
  source 1..12: mol_type = genomic DNA
gagttctttatt